(19) United States
(12) Patent Application Publication
(10) Pub. No.: US 2005/0214367 A1
(43) Pub. Date: Sep. 29, 2005

(54) Processes for making coated phytochemicals and tocopherols and products formed therefrom

(76) Inventors: Volker Kuellmer, Decatur, IL (US); Rishi Shukla, Decatur, IL (US)

Correspondence Address: KIRKPATRICK & LOCKHART NICHOLSON GRAHAM LLP;HENRY W. OLIVER BUILDING, 535 SMITHFIELD STREET, PITTSBURGH, PA, 15222, US

(21) Appl. No.: 11/086,946

(22) Filed: Mar. 22, 2005

Related U.S. Application Data
(60) Provisional application No. 60/555,197, filed on Mar. 22, 2004.

(51) Int. Cl. A61K 9/20

(52) U.S. Cl. 424/464; 264/109

(57) ABSTRACT

Methods and processes are provided herein for coating tocopherol succinate and phytoestrogen, e.g., an isoflavone. The methods include dispersing tocopherol succinate or phytoestrogen (e.g., an isoflavone) in a solvent and a coating composition, and then drying the composition. Coated tocopherol succinate and phytoestrogens (e.g., isoflavones) are also provided.

### CROSS-REFERENCE TO RELATED APPLICATIONS

This application claims the benefit of U.S. Provisional Application No. 60/555,197, filed on Mar. 22, 2004, the entire teachings of which are incorporated herein by reference.

### BACKGROUND

Plant materials are known to contain a number of classes of organic low molecular weight bioactive compounds such as phytoestrogens. Historically these phytochemical compounds have been considered to be non-nutritive, however, recent scientific evidence suggests that some of these compounds, such as phytoestrogens, may play an important role in the maintenance of health, in chemoprevention, and in the mitigation of certain conditions or diseases associated with the circulation of sex hormones, including sleep disorders and vaginal dryness.

Edible plants normally contained in the diet, or materials used as herbal remedies/dietary supplements, may contain collections of certain structurally related bioactive phytochemicals. These related compounds are often unique in their amounts and distribution when compared among various plant sources. Some of the most notable groups of related phytochemicals exhibiting bioactivity include, but are not limited to, flavonoids, isoflavones, saponins, lignans (plant compounds closely related to lignins), alkaloids, catechins and phenolic acids.

Epidemiology studies relating diet to disease suggest that dietary components may cause a reduced risk of certain diseases in some populations. For instance, far eastern populations consuming soy as a staple have reduced rates of breast, colon and prostate cancers and coronary heart disease, while populations in Finland have reduced rates of prostate cancer. Researchers are just now studying specific dietary compounds in order to understand the basis for these epidemiological observations.

Among the various plants consumed in the diet, several are rich sources of beneficial phytochemicals. Soy products contain high amounts of isoflavones and saponins. Unrefined grains including, without limitation, wheat, psyllium, rice, flax and oats, all contain lignans, which are compounds closely related to lignins. Cocoa contains phenolic acids and catechins, which also occur in green teas. Certain non-dietary plants are also sources of these same chemical molecules, such as lignans and isoflavones in kudzu root or red clovers. Isoflavones and lignans act as weak estrogenic substances. Tea plants are also a rich source of phytochemicals, including catechins and phenolic acids.

It has been proposed to use isoflavones to treat or prevent breast cancer, prostate cancer, skin cancer, and colon cancer. It also has been proposed that use of isoflavones may reduce or prevent various symptoms related to the onset and duration of menopause, including “hot flashes” and osteoporosis, and they may be effective in certain cardiovascular applications, including prevention of heart disease, reducing cholesterol-lipid levels, modulating angiogenesis, and other advantageous vascular effects. Moreover, isoflavones have been implicated in reducing headaches, dementia, inflammation, treating alcohol abuse, and may play a role in immunomodulation.

Soy, as well as other plants such as red clover, contains a variety of beneficial isoflavones. Non-limiting examples of specific isoflavones found in soy protein include: the glucosides such as genistin, daidzin and glycitin, and the aglycones such as genistein, daidzein and glycitein. Red clover contains these isoflavones along with biochanin A and formononetin. Ingestion of soy protein specifically has been shown to significantly reduce menopausal symptoms such as “hot flashes,” although not to the extent of conventional estrogen replacement therapy. Soy protein also appears to be useful in treating cyclical mastalgia (breast pain associated with the menstrual cycle). Soy isoflavones are also antioxidants and increase bone density, and soy protein containing higher levels of isoflavones has been shown to have a lipid-lowering effect. Although conventional pharmaceuticals can be used to obtain all of these effects, soy protein is often sought after by those seeking a “natural” alternative to hormone replacement therapy, calcium supplements, cholesterol-lowering drugs, and to prevent cancer.

People who eat a high-soy diet show reduction of many of these above-discussed symptoms. This suggests that ingesting a combination of certain phytochemicals in a ratio such as found in soy may result in an additive or synergistic effect. However, a high soy diet has some undesirable effects, including flatulence and undesirable taste, and Western consumers are characteristically hesitant to change their lifestyle to incorporate soy in their diets, even for such benefits.

Isoflavones, which are sometimes referred to as phytoestrogens, are heterocyclic phenols, and are understood to include the soy compounds genistin, daidzin and glycitin, as well as biochanin A, equol, formononetin, and o-desmethylangolensin and natural derivatives thereof. These compounds and their aglycone or de-methylated aglycone forms, such as genistein, daidzein and glycitein, are believed to all have similar activities once they are ingested.

These various compounds can be ingested by consumption of plant material, but the amount may be limited by the quantity of plant material that can be consumed by an individual. The compounds can be extracted and concentrated, but they must then be put in a form for direct consumption by the individual (e.g., tabletting as dietary supplements) or incorporated into a food item.

The concentrated compound must in a form that can be easily handled, preferably as a free-flowing powder. Many bioactive plant compounds are difficult to produce in such a form. For instance, compounds that have a lipophilic component, that is, are oily, are difficult to formulate as a free flowing powder, and usually assume a clumping or pasty form.

Products that contain phytoestrogens, and are marketed in bulk quantities often exhibit poor flow characteristics. These can be caused by such phenomena as interparticle ionic charge, or by the pick-up of moisture by hygroscopic components in the particles, such as sugars. In addition to poor flow characteristics, such phenomena also can result in decreased shelf life and difficulties in handling and product preparation. Furthermore, the high carbohydrate content of phytoestrogen isoflavone products makes such products particularly difficult to tablet.

There is therefore a need for improved methods for coating such phytochemicals so that they can be easily tabletted and included in formulations such as processed foods.

### SUMMARY OF THE INVENTION

Processes are provided for coating a tocopherol succinate or a phytoestrogen, such as an isoflavone, or a phytoestrogen-containing compound. Tocopherol succinate and phytoestrogens coated by the methods of the present invention, such as coated isoflavones, are also provided.

“Phytoestrogen” and “phytoestrogen-containing compound” are used interchangeably herein, and are intended to include compounds that are substantially pure phytoestrogen, or compounds which contain phytoestrogen as a substantial component, such as plant extracts wherein phytoestrogen has been concentrated, relative to unprocessed fresh or dried plant material. One example is NOVASOY®, a soybean extract that contains approximately 40% phytoestrogen.

According to one embodiment, the invention provides a process for producing a coated tocopherol succinate or coated phytoestrogen composition, comprising: (a) dispersing a binder composition in a solvent to form a binder solution; (b) passing tocopherol succinate or phytoestrogen composition in powder form through the binder solution, where the binder solution is in an atomized state, to produce wetted tocopherol succinate or wetted phytoestrogen composition; (c) passing the wetted tocopherol succinate or wetted phytoestrogen composition through a region of turbulent gas to form agglomerated tocopherol succinate or agglomerated phytoestrogen composition; and (d) evaporating the solvent from the agglomerated tocopherol succinate or agglomerated phytoestrogen composition; thereby forming dried coated tocopherol succinate or dried coated phytoestrogen composition.

The process can further include screening the dried coated tocopherol succinate or the dried coated phytoestrogen composition. Particles over 20 mesh in size can be removed. The binder composition can be cellulose, cellulose derivatives, maltodextrin, alginic acid derivatives, calcium lactate, gum arabic, gelatin, sugar, sugar alcohols, glycerol, starch, modified starch, pregelatinized starch, polyvinylpyrrolidones, stearic acid, gum acacia, and hydrogenated vegetable oil, or mixtures thereof. The cellulose derivative can be ethylcellulose, methylcellulose and hydroxypropylmethylcellulose or mixtures thereof. The solvent can be water, C1-C3 alcohol, and a mixture of water and C1-C3 alcohol. The alcohol can be ethanol, isopropanol, methanol, or a mixture thereof. The solvent can be evaporated by a fluid bed dryer.

The coated tocopherol succinate or coated phytoestrogen composition can be a free-flowing powder. The binder solution can be about 0.5% to about 10.0% hydroxypropylmethylcellulose in water, or can be about 3.0% to about 4.0% hydroxypropylmethylcellulose in water.

The phytoestrogen can be an isoflavone. The isoflavone can be genistein, daidzein, glycitein, biochanin A, equol, formononetin, and o-desmethylangolensin, or natural derivatives of genistein, daidzein, glycitein, biochanin A, equol, formononetin, and o-desmethylangolensin, or chemical derivatives of genistein, daidzein, glycitein, biochanin A, equol, formononetin, and o-desmethylangolensin.

In another embodiment, the invention includes a coated tocopherol succinate or a coated phytoestrogen composition made by the above processes.

The coating composition can be any food-grade compound useful as an edible coating. Such coatings are generally dissolved or suspended in a liquid and/or solvent, mixed with the compound to be coated, and then dried so that the coating dries in a layer around the compound. The compound particles do not need to be perfectly or completely coated, it is only necessary that the compound be coated sufficiently to attain the desired characteristics, e.g., that the compound become a free-flowing powder. Non-limiting examples of coating compositions useful in the present invention include cellulose, cellulose derivatives, maltodextrin, alginic acid derivatives, calcium lactate, gum arabic, gelatin, sugar, sugar alcohols, glycerol, starch, modified starch, pregelatinized starch, polyvinylpyrrolidones, stearic acid, gum acacia, hydrogenated vegetable oil, and mixtures thereof. Non-limiting examples of useful cellulose derivatives include ethylcellulose, methylcellulose, hydroxypropylmethylcellulose, and mixtures thereof.

The solvent can be any liquid in which the coating composition can be dissolved or suspended. Non-limiting examples of useful solvents include water, alcohol, and water/alcohol mixtures. Non-limiting examples of alcohols that are useful as solvents in the present invention include ethanol, isopropanol, methanol, and mixtures thereof.

After the coating composition is mixed with the solvent, and the compound to be coated is added, the coated compound is then dried. The drying can be done by a spray dryer, a fluid bed dryer, or by a combination of spray dryer and fluid bed dryer.

Non-limiting examples of a phytoestrogen with which the present invention may be use is isoflavone. Examples of isoflavones which may be processed according to the present invention include genistein, daidzein, glycitein, biochanin A, equol, formononetin, o-desmethylangolensin, and mixtures thereof. The isoflavone can also include natural derivatives of these compounds, and chemically-synthesized versions of these compounds.

According to another embodiment of the present invention, a process is provided for producing a coated phytoestrogen composition, wherein the process includes: uniformly dispersing a coating composition in a solvent to form a mixture; uniformly dispersing a phytoestrogen into the mixture; and drying the product to form dry particles to thereby produce a coated phytoestrogen composition.

The present invention also is directed to coated phytochemicals and tocopherol made by a process according to the present invention, and to articles of manufacture that include the coated tocopherols and phytochemicals made by the process.

The coated compositions made by the process of the present invention have the advantage of being a free-flowing powder. Inclusion of ingredients in a tablet form is made significantly easier by use of free-flowing powders, as opposed to ingredients that are sticky or clump. Use of a free-flowing powder also makes easier the incorporation of the compositions into other products including, but not limited to, processed foods, fortified baking mixes, food bars, snack products, beverages, beverage powders, etc. The coating process of the present invention allows controlled concentrations of phytoestrogens and tocopherols to be incorporated into tablets and food products, relative to uncoated phytoestrogens and tocopherols.

These and other characteristics and advantages of embodiments of the invention will be apparent to those of ordinary skill upon considering the following detailed description of certain non-limiting embodiments of the present invention.

### DETAILED DESCRIPTION

Methods and processes are provided herein for coating a tocopherol succinate or phytoestrogen-containing product, such as, but not limited to, a coated isoflavone-containing product. The methods include dispersing a powder containing the tocopherol, phytoestrogen or a product containing the same in a solvent with a coating composition to produce a mixture, and then drying the mixture. Specifically, the powder containing the tocopherol succinate or phytoestrogen is passed through atomized binder solution, then through a region of turbulent gas to form agglomerates, which are then dried. Coated tocopherol succinate and phytoestrogens such as coated isoflavones, are also provided.

A number of patents (see, for example, U.S. Pat. Nos. 3,914,430; 5,925,381; 5,938,990; 6,001,554; 6,146,825; 6,130,343; and 6,150,086) describe various aspects of multiple micro-encapsulation systems for oleophilic (fat- and oil-soluble) substances, particularly the fat-soluble vitamins, which includes incorporating an oleophilic substance into a primary polymer, typically methylcellulose and hydroxypropyl methylcellulose. These patents are described in further detail below. None of these, however, provides compositions for encapsulated soy protein or other beneficial soy compounds.

U.S. Pat. No. 6,162,474 describes a powder composition that includes droplets of a fat soluble vitamin dispersed in a modified polysaccharide matrix, such as modified starch.

U.S. Pat. No. 6,139,872 describes a process for making a nutrient supplement powder by forming a plastic mass, extruding the material, then cooling and comminuting it.

U.S. Pat. No. 6,030,645 discloses a flowable dry particle containing an active oleophilic substance in a matrix of a carrier material and a coating of calcium silicate.

International Patent Publication No. WO 97/38016 discloses cellulose esters that may be designed to dissolve under specific conditions and that may be used as coatings for controlled-release applications.

U.S. App. Pub. No. U.S. 2001/0009679 A1 discloses a powder composition containing at least one fat-soluble vitamin dispersed in a matrix of a natural polysaccharide gum or a mixture of gums having an emulsifying capacity and/or a protein or a mixture of proteins having an emulsifying capacity.

International Patent Publication No. WO 01/80823 A2 discloses sol-gel microcapsules of inorganic polymers useful for the topical delivery of sensitive active ingredients.

U.S. Pat. No. 6,130,343 describes a method for producing a tocopheryl succinate powder by contacting a pharmaceutically acceptable binder in a fluidized bed and evaporating solvent from the tocopheryl succinate in the said fluidized bed, where the drying and coating are performed together in a fluidized bed. One of the drawbacks of this method is incomplete coating of the raw material during the drying phase.

U.S. Pat. No. 3,914,430 describes a spray drying process for preparing coated free-flowing high density agglomerates including by introducing ultra-fine particle size adsorbents into a spray chamber along with alpha-tocopherol. The agglomerate pass through a drying zone without the wall adhesion and wet mass accumulation observed in certain conventional processes.

U.S. Pat. No. 4,767,217 describes a method and apparatus for mixing two substances under sanitary conditions in which a solid particulate substance is introduced into an inlet chamber with radial and downward velocity components and is mixed with a second substance in a mixing chamber below the inlet chamber by rotating a vertical shaft with mixing blades thereon in the mixing chamber.

It appears that a coated phytoestrogen/isoflavone product is not commercially available. Due to the flow problems and shelf-life issues discussed above, the present inventors recognized that a need exists in the art for a commercially useful process for coating phytoestrogens, and particularly the isoflavones. Such coated phytoestrogen products would be useful for incorporation into nutraceutical supplements and nutritional products, including, for example, supplement tablets, cereals, energy bars, nutritional drinks, and ingredients and mixes for making such products.

Therefore, embodiments of the present invention are directed to a free-flowing particulate product containing tocopherol succinate or phytoestrogen, produced by a process of coating, microencapsulation and agglomeration. As an example, an embodiment of the present invention involves the coating and agglomeration of a powder material by mixing the material with a coating composition, such as a polymer, in a solvent, followed by drying in a fluidizer. The mixing can be done in a mixer used for mixing solids with other solids and/or liquids under sanitary conditions. The polymer, the solvent, and the process parameters used in the mixer are all selected and/or manipulated by the skilled practitioner to produce a coated phytoestrogen end product having the desired particle size distribution and characteristics for a particular end use.

In one embodiment, tocopherol succinate or soy particles containing isoflavones are coated and agglomerated using a water soluble cellulose derivative (such as methylcellulose and/or ethylcellulose) in an alcohol or alcohol/water solvent. The coating and agglomeration may be performed in a mixing system adapted for sanitary mixing of the product being coated with a pharmaceutically acceptable coating or binder. The mixing system may utilize air pressure and evaporation temperatures in suitable ranges to provide the desired particle size/agglomeration characteristics and the desired amount of coating on the particles.

Various coating compositions are useful in the practice of the invention. Polymers and other coating substances suitable for use as coating compositions in the present invention include, but are not limited to, cellulose, water soluble cellulose derivatives (methylcellulose and hydroxypropylmethylcellulose, for example), maltodextrin, alginic acid derivatives, calcium lactate, gum arabic, gelatin, sugar, sugar alcohols, glycerol, modified starches, pregelatinized starches, polyvinylpyrrolidones, stearic acid, gum acacia, and hydrogenated vegetable oil. The preferred coating compositions in the present invention are the water soluble cellulose derivatives. Those of skill in the art will recognize that particular coatings may be more or less applicable to use in a particular application. Based on the present disclosure and the level of skill in the art, one of ordinary skill will be able to modify the teachings herein and practice the invention in its various embodiments without undue experimentation.

By way of example, two coating compositions, METHOCEL® (methylcellulose) and ETHOCEL® (ethylcellulose) (Dow Chemical Co., Midland, Mich.), in their various forms, can produce coatings having various characteristics, including particle color, particle size, particle coarseness, etc., as well as having potential applicability in particular end uses. Furthermore, the various process parameters disclosed below will affect certain characteristics of the coated, agglomerated particulate end product. Again, those of ordinary skill in the art will be able to use the teachings herein to modify and adapt the present invention for the production of coated and agglomerated phytoestrogen-containing products having applicability to appropriate end uses without undue experimentation. The more important aspects in the practice of the present invention are selection of the appropriate coating compositions for the application in mind, as discussed above, and selection of the appropriate solvent, as discussed further below.

Choice of solvent will, of course, vary depending upon the polymer chosen and the polymer's solubility in water and/or organics, the size and/or viscosity of the polymer chosen, etc. Particularly preferred solvents allow for control of drying/evaporation so that resulting dry particles having desirable characteristics can be produced. For example, alcohols and/or alcohol/water solvents are preferred when using a polymer that is a water soluble cellulose derivative such as methylcellulose or ethylcellulose. A particularly preferred alcohol for such an embodiment is ethanol, and even more preferred are ethanol/water mixtures, at various ratios (depending primarily upon the solubility of the polymer in water and/or the size and/or viscosity of the polymer). For example, in certain embodiment employing ETHOCEL® as the polymer, a 100% ethanol solvent may be preferred, while in certain embodiments employing METHOCEL® as the polymer, a mixture of 80% w/w ethanol:20% w/w water may be preferred. Again, the selection of solvent will depend upon, for example, the size/viscosity of the chosen polymer and the desired characteristics of the coated, agglomerated end product.

In order to become free-flowing and dustless, finely-sized powders must be agglomerated. Agglomeration is also used to increase the size of the powder particles.

There are two types of agglomerators, compaction agglomerators and noncompaction agglomerators. Compaction agglomerators use mechanical pressure crush powder particles together to form larger particles. Binders may or may not be used.

Noncompaction agglomerators keep the powder in motion while it is sprayed with the binder in solution. The binder holds groups of smaller particles together, forming agglomerates of the particles.

Rotary drum agglomerators rotate so that the particles are constantly falling, and a nozzle within the drum sprays the binder onto the particles. Many drum agglomerators have internal appendages to better distribute the particles. Rotary drum agglomerators can be inexpensive and cheap to operate, but can require relatively long residence times.

A fluid bed agglomerator blows gas up through a perforated conveyer. The powder is fed onto the conveyer, and the particles are suspended in a constantly circulating layer by the upwardly-blowing gas. The binder solution may be sprayed down onto the layer of particles in the fluidized bed. As the fluidized layer of particles moves down the conveyer, the particles bind together to form agglomerates. The agglomerates can be dried by use of warmed gas in the bed.

Another type of noncompaction agglomerator is the vertical continuous agglomerator. The powder to be agglomerated is fed in the top, and falls through the binder solution, which is injected into the agglomerator via an atomizer. The wetted powder particles then fall into a region of turbulence, caused by blades rotating rapidly on a shaft. The turbulence causes intense mixing of the binder-wetted powder particles, which form agglomerates. The size of the agglomerates formed depends on a number of factors, including the speed of the rotating blades (faster speed produces smaller agglomerates), and the amount of binder solution used (more moisture produces larger agglomerates), and the concentration of the binder solution (e.g., increased concentrations on methylcellulose produce smaller agglomerates). The advantage of this type of agglomerator is that the particles have a residence time of only a few seconds, and flow through the system, and therefore production, is continuous. The agglomerates are also more completely coated than with other methods.

After passing through the area of turbulent gas, the agglomerates are dried, for instance, the agglomerates can be allowed to fall to a fluid bed dryer.

The mixer system and equipment used to carry out the coating/agglomeration process of the invention can ultimately be left to the practitioner's choice. One system that can be used is the Hosokawa Bepex Schugi Flexomix Model FX-100 system (referred to as the “Schugi” of the following Examples) with a fluid be dryer Model FBS-5 (Hosokawa Bepex; 333 N.E. Taft Street, Minneapolis Minn. 55413). Alternative mixing systems available include, for example, those sold by Glatt Air Techniques Inc., Ramsey, N.J.

As noted above, certain process parameters of the present invention can be manipulated in order to control final particle size/characteristics. For example, particle size (the amount of agglomeration) can in part be controlled by adjustment of the evaporation temperatures and/or by adjustment of nozzle air pressures (higher pressures producing smaller particles). As previously noted, the polymer and its physical characteristics (particularly its size) will also have an impact on particle size.

The amount and thickness of coating applied can be varied depending upon the ultimate goal of the coating process. Coating thickness can be controlled through, for example, variation of amount of polymer used, process parameters, or by application of additional layers of the same or other polymers, as desired by the practitioner.

The coated particles of the invention are suitable for use in their free-flowing form, or they may also be tabletted using any number of art-recognized tabletting processes and formulations. Such tablets are typically consumed as health/nutritional supplements. The free-flowing coated particles can be used in food or dairy applications, as well as in various health/nutraceutical formulations and applications.

### EXAMPLES

### Example 1

### Coating of Succinate and Isoflavone with ETHOCEL®

The following example describes a process for separately coating vitamin E (succinate) and isoflavone (NOVASO® isoflavone, a powdered compound extracted from soybeans, available from Archer Daniels Midland, Decatur, Ill., USA) with an ETHOCEL® solution. Agglomeration and drying are performed separately, which provides for a more uniform coating of the product during the agglomeration process, and more uniform drying as well, relative to previous methods where drying and coating are done together in a fluidized bed. The coated agglomerated particles dried to produce a free flowing granular product. Results are attached in Table 1 and Table 2.

Description of Feed: Approximately 100 kg of succinate (vitamin E) was received for testing. The dry powder was light tan in color and had a waxy/oily feel to it. It had a mean particle size of about 200 mesh, a moisture content of 0.06% by weight and a loose bulk density of 0.38 g/cc. During shipping, the powder had caked inside the drums and was therefore passed through a mill before feeding to the Schugi in order to break up the lumps.

NOVASOY® is a powdered compound extracted from soybeans, and is about 40% isoflavones. It also contains soy saponins and protein. The original powder is very fine and at times dusty, with poor flow and tabletting characteristics. It sticks to surfaces and tends of cause bridging when flowing.

The binder solution consisted of ethylcellulose (Dow ETHOCEL® Standard 45 Premium) dissolved in denatured ethanol at concentrations of both 4% and 6% by weight. With agitation, the two binder solutions took 30-45 minutes to completely dissolve.

Test Set-Up:
Disintegrator Model RP-8-K115
Small Pump Package (with piston pump and tank with load cell and agitator)
K-Tron Single Screw loss-in-weight feeder
AccuRate Screw Feeder
Schugi Flexomix Model FX-100 mixer
Fluid Bed Dryer Model FB-5
30″ Sweeco Screener w/20 Mesh Screen

The Disintegrator was used to delump the fineset succinate feed prior to processing through the Schugi. A ⅛″ screen was used along with a rotor speed of 3600 rpm.

In separate trial, the powdered materials were fed to the Schugi using a loss in weight (LIW) screw feeder. A loss-in-weight (LIW) feeder measures the weight loss of a dry bulk material in an integral hopper as the material is dispensed to a process. This weight information is used to control the feedrate. The binder solution was added through either two, three or four two-fluid nitrogen atomized spray nozzles. The resulting granules were discharged directly into the first zone of the Fluid Bed dryer where they were dried in either a batch or continuous fashion.

Test Work: An objective of the trials was to coat the succinate with the polymer binder to produce a free flowing, less waxy feeling granule that would perform well in a tabletting press. In connection with the trial of coating the NOVASOY® soy particles, an objective was to turn the fine soy powder into free-flowing, less dusty granules also suitable for tabletting. The finished products were to have a maximum polymer coating of 2% by weight with the majority of the granules in the 60 to 100 mesh range. The objective was for approximately 10% of the final product to be able to pass through a 200 mesh screen and 0% to be retained on a 20 mesh screen. In each trial the target product density was 0.5-0.6 g/cc.

For all of the testing, the blades of the Schugi were angled at +5° for slight conveyance of the material through the mixer. Atomizing pressure on the nozzles was set at 65 psi to ensure that the somewhat viscous binder would be atomized properly. The powder rate through the agglomerator was fixed at 240 lb/hr to create a full and efficient mixing environment.

The first five runs processed the succinate using a Schugi rotor speed of 3000 rpm which was found to produce the best results during prior trials. Runs 1 and 2 were made using the relatively fine material.

At first, a total of four spray nozzles were used in the Schugi. During the first run, the powder feed (the raw material) backed up upstream of the nozzles due to the stickiness of the powder and the narrow feed opening. For the next two runs (runs 2 and 3), the nozzle at the feed opening was removed to create a larger space for the powder to fall through. As the succinate runs progressed (runs 4 and 5), less and less binder was added and the number of spray nozzles was reduced to two. These were located 180° apart from one another (in the second and fourth opening after the feed inlet) to keep the spray patterns from overlapping, which can create a high liquid concentration in one spot during mixing, possibly leading to lump formation.

The first four runs were small batch runs wherein the agglomerated succinate granules were dried using inlet temperatures in the Fluid Bed dryer of 110-130° F. and were held in the dryer until the granules reached a temperature of 80° F. The final run of the first five runs was a longer continuous run, which processed all of the remaining succinate using the conditions of run 4 (0.6% ETHOCEL® coating level).

Runs 6 through 9 processed the NOVASOY® material using three spray nozzles. In run 6 the rotor speed of the Schugi was set at the same 3000 rpm as with the succinate. This produced a product that was finer than desired. For the next run (run 7), the rotor speed was increased to 4300 rpm and the binder solution application rate was raised slightly, providing a 1.9% ETHOCEL® coating. This was an improvement, but the wet agglomerates still were considered to be on the fine side.

In run 8, the concentration of the binder solution was reduced from 6% by weight polymer/solvent to 4% so that the soy agglomerates were made wetter and the binder coated the particles more evenly, thereby making them stronger when dried. The last run (run 9) was made processing the remaining soy material under the run 8 conditions (1.3% coating levels).

The dried products from each of the runs were screened on a 20 mesh screen and each fraction was packaged and weighed.

Test Results: Using binder concentrations of 4% and 6% ETHOCEL®, the succinate was coated with between 1.6% and 0.8% ETHOCEL® by weight in the finished product. In all of the samples generated, the product was much more free flowing and less tacky than the original feed powder. The particle size of the final product was mostly larger than 100 mesh, with up to 18% by weight retained on a 20 mesh screen. Very little passed a 200 mesh screen. Product densities were 0.31-0.35 g/cc, and the moisture content of the dried granules was 0.13% or less. A rotor speed of 3000 rpm seemed to produce the most uniform granulation and the least amount of wall buildup.

The isoflavone powder was agglomerated and coated with a maximum of 2% w/w Ethocel, resulting in a free-flowing product which was directly compressible. The particle size was smaller that the agglomerated succinate, and a minimum of 90% passed through a 40 mesh screen and about 20% passed a 200 mesh screen. A rotor speed of 4300 rpm produced the desired granulation in the product. The product bulk density was about 0.30 to about 0.35 g/cc. The results are shown in Tables 1-5, below.

TABLE 1
A. Schugi Agglomeration System

| Solid #1 Name | | Tocopherol Succinate (Vitamin E); “TS” | | | |
|---|---|---|---|---|---|
| Solid #2 Name | | Novasoy (Isoflavone); “NS” | | | |
| Liquid Name | | Ethocel/Ethanol Solution | | | |
| Screen Area (ft²) | | 2.02 (First Zone) | | | |
| | Run No.: | | | | |
| | 1 | 2 | 3 | 4 | 5 |

B. Schugi Data; Model No. FX-100

| | | | | | |
|---|---|---|---|---|---|
| Run Time: | | | | | :25 |
| Schugi Rotor Speed (rpm) | 3000 | 3000 | 3000 | 3000 | 3000 |
| Schugi Motor Load (amps) | | | | | |
| Knife Position | +5° | +5° | +5° | +5° | +5° |
| Nozzle Setup (type & qty.) | N13 (4) | N13 (3) | N13 (3) | N13 (2) | N13 (2) |
| Nozzle Gas Pressure (psi) | 65 | 65 | 65 | 65 | 65 |
| Powder Feed Rate (lb/hr) | 240 | 240 | 240 | 240 | 240 |
| Powder Feed Type | TS | TS | TS | TS | TS |
| Liquid Feed Rate (lb/hr) | 54 | 48 | 33 | 24 | 25 |
| Binder Solution Type | 6% | 6% | 6% | 6% | 6% |
| | Ethocel/EtOH | Ethocel/EtOH | Ethocel/EtOH | Ethocel/EtOH | Ethocel/EtOH |
| Ethocel Loading in | 1.3 | 1.2 | 0.8 | 0.6 | 0.6 |
| finished product (wt/wt %) | | | | | |

C. Fluid Bed Data; Model No. FBS-5.0

| | | | | | |
|---|---|---|---|---|---|
| Inlet Air Temp(° F.) | 109 | 116 | 129 | 115 | 126/131/111 |
| Bed Temp(° F.) | 80 | 80 | 81 | 80 | 50/93/96 |
| Air Out Temp(° F.) | | | 76 | 76 | 68 |
| Air Flow Reading (in. H₂O) | | | 0.5 | 0.5 | .5/.5/.05 |
| Total Pressure(in H₂O) | | | | | |
| Screen Size (A-H, 50) | RAR | RAR | RAR | RAR | RAR |
| | Conidur | Conidur | Conidur | Conidur | Conidur |
| Fluidizing Velocity | | | | | |
| (AFPM) | | | | | |

D. Material Collected (lb)

| | | | | | |
|---|---|---|---|---|---|
| Over Size +20 Mesh | | 5.05 | 7.45 | 3.55 | 8.90 |
| On Size −20 Mesh | | 13.15 | 11.75 | 16.05 | 27.95 |
| | | | | | FB Prod |
| | | | | | 228 lb/hr |
| FOA: | 0.01 | 0.01 | 0.01 | 0.01 | 0.01 |
| Vs(ft/min): | | | 3003.85 | 2967.94 | |
| gas density | 0.068927944 | 0.068090278 | 0.066587436 | 0.068208696 | |
| (lb/ft3): | | | | | |

With reference to Table 2 below, drying NOVASOY® soy coated with 4% ETHOCEL® binder using an inlet air temperature of about 150-160° F. and a fluid bed temperature of 100° F. allowed for continuous processing, and resulted in a product with less fines, lower moisture content, more uniform granulation, and the least amount of wall buildup relative to other runs at other settings. The majority of the particles were greater than 100 mesh.

TABLE 2
A. Schugi Agglomeration System

| Solid #1 Name | Tocopherol Succinate (Vitamin E); “TS” |
|---|---|
| Solid #2 Name | Novasoy (Isoflavone); “NS” |
| Liquid Name | Ethocel/Ethanol Solution |
| Screen Area (ft²) | 2.02/2.02/1.01 |

| Run No.: | | | |
|---|---|---|---|
| 6 | 7 | 8 | 9 |

B. Schugi Data; Model No. FX-100

| | | | | |
|---|---|---|---|---|
| Run Time: | | | | |
| Schugi Rotor Speed (rpm) | 3000 | 4300 | 4300 | 4300 |
| Schugi Motor Load (amps) | | | | |
| Knife Position | +5° | +5° | +5° | +5° |
| Nozzle Setup (qty.) | N13 (3) | N13 (3) | N13 (3) | N13 (3) |
| Nozzle Gas Pressure (psi) | 65 | 65 | 65 | 65 |
| Powder Feed Rate (lb/hr) | 240 | 240 | 240 | 240 |
| Powder Feed Type | Novasoy | Novasoy | Novasoy | Novasoy |
| | 152400 | 152400 | 152400 | 152400 |
| Powder Feed Lot# | 203221 | 203221 | 203221 | 203221 |
| Liquid Feed Rate (lb/hr) | 69 | 76 | 82 | 82 |
| Binder Soln Type | 6% | 6% | 4% | 4% |
| | Ethocel/EtOH | Ethocel/EtOH | Ethocel/EtOH | Ethocel/EtOH |
| Ethocel Loading (wt/wt %) | 1.7 | 1.9 | 1.3 | 1.3 |

C. Fluid Bed Data; Model No. FBS-5.0

| | | | | |
|---|---|---|---|---|
| Inlet Air Temp(° F.) | 115 | 140 | 156 | 150-160 |
| Bed Temp(° F.) | 80 | 80 | 80 | 100 |
| Air Out Temp(° F.) | | | 72 | |
| Air Flow Reading (in. H₂O) | <.5 | <.5 | <.5 | <.5 |
| Total Pressure(in H₂O) | 2 | 2 | 2 | 2 |
| Screen Size (A-H, 50) | RAR Conidur | RAR Conidur | RAR Conidur | RAR Conidur |
| Fluidizing Velocity (AFPM) | | | | |

D. Material Collected (lb)

| | | | | |
|---|---|---|---|---|
| Over Size +20 Mesh | 3.30 | 2.40 | 3.45 | 13.35 |
| On Size −20 Mesh | 9.45 | 9.85 | 10.40 | 66.35 |

TABLE 3
A. Schugi Agglomeration System

| | |
|---|---|
| Solid #1 Name | Succinate Fines |
| Solid #2 Name | |
| Liquid Name | |
| Screen Area (ft²) | 2.02 -Z1, Z2 1.03- Z3 |
| | Overflow Weir Z1/Z2/Z3: 8″/8″/8″ |
| | Underflow Weir Z1/Z2/Z3: All Closed |
| Screen Code Z1/Z2/Z3: | RAR Conidur |

| Run No.: | | | | |
|---|---|---|---|---|
| 1 | 2 | 3 | 3 | 4 |

B. Schugi Data; Model No. FX-100

| Run Time (Min): | | | 13:41 | 14:24 | 8:40 |
|---|---|---|---|---|---|
| Schugi Rotor Speed (rpm) | 3000 | 3000 | 3000 | 3000 | 3000 |
| Schugi Motor Load | | 1.1 | 1.1 | 1.1 | 1.1 |
| (amps) 1.1 | | | | | |
| Knife Position | +5° | +5° | +5° | +5° | +5° |
| Nozzle Setup | N13 (3) | N13 (3) | N13 (3) | N13 (3) | N13 (3) |
| Nozzle Air Pressure (psi) | 65 | 65 | 65 | 65 | 65 |
| Feed Lot | SF | SF | SF | SF | SF |
| | 61043067 | 61043067 | 61043067 | 61043067 | 61043067 |
| Powder Feed Rate (lb/hr) | 240 | 240 | 200 | 200 | 200 |
| Liquid Binder | 6% Ethocel | 6% Ethocel | 6% Ethocel | 6% Ethocel | 6% Ethocel |
| Liquid Feed Rate (lb/hr) | 40 | 33 | 27 | 29.7 | 32 |
| Coating Level (%) | 0.99 | 0.82 | 0.80 | 0.88 | 0.95 |

C. Fluid Bed Data; Model No. FBS-5.0

| | | | | | | |
|---|---|---|---|---|---|---|
| Inlet | Zone 1 | 127 | 120 | 131 | 132 | 123 |
| Air | Zone 2 | 141 | | 126 | 128 | 131 |
| Temp (° F.) | Zone 3 | 117 | | 97 | 123 | 113 |
| Bed | Zone 1 | 51 | 90 | 63 | 62 | 55 |
| Temp | Zone 2 | 86 | | 98 | 96 | 93 |
| (° F.) | Zone 3 | 92 | | 86 | 97 | 95 |
| Air Out | Zone 1 | | | | | |
| Temp | Zone 2 | 64 | | 65 | 68 | 68 |
| (° F.) | Zone 3 | | | | | |
| Air Flow | Zone 1 | | | | | |
| Reading | Zone 2 | | | | | |
| (in. H₂O) | Zone 3 | | | | | |
| Total | Zone 1 | 2.5 | | 4 | 4.5 | 4 |
| Pressure | Zone 2 | 2 | | 2 | 2 | 2.5 |
| (in. H₂O) | Zone 3 | | | | | |
| Fluidizing | Zone 1 | 0 | 0 | 0 | 0 | 0 |
| Velocity | Zone 2 | 0 | 0 | 0 | 0 | 0 |
| (AFPM) | Zone 3 | 0 | 0 | 0 | 0 | 0 |

D. Material Collected (lb)

| | | |
|---|---|---|
| Over Size | lb/hr | 150 |
| (+20 M) | | |
| On Size | lb/hr | |
| (−20 M) | | |
| Baghouse | lb/hr | 50 |
| Fines | | |

| | | | | | |
|---|---|---|---|---|---|
| FOA: | | | | | |
| Vs(ft/min): | 0.00 | 0.00 | 0.00 | 0.00 | 0.00 |
| | 0.00 | 0.00 | 0.00 | 0.00 | 0.00 |
| | 0.00 | 0.00 | 0.00 | 0.00 | 0.00 |
| gas | 0.06681431 | 0.06762069 | 0.066362098 | 0.06625 | 0.067272727 |
| density(lb/ft3): | | | | | |
| | 0.065257903 | 0.08526087 | 0.066928328 | 0.06670068 | 0.066362098 |
| | 0.06797227 | 0.08526087 | 0.070412926 | 0.067272727 | 0.068446771 |

TABLE 4
A. Schugi Agglomeration System

| | |
|---|---|
| Solid #1 Name | Succinate Fines |
| Solid #2 Name | |
| Liquid Name | |
| Screen Area (ft²) | 2.02 -Z1, Z2 1.03- Z3 |
| | Overflow Weir Z1/Z2/Z3: 8″/8″/8″ |
| | Underflow Weir Z1/Z2/Z3: All Closed |
| Screen Code Z1/Z2/Z3: | RAR Conidur |

| Run No.: | | | | |
|---|---|---|---|---|
| 4 | 4 | 5 | 6 | 7 |

B. Schugi Data; Model No. FX-100

| | | | | | |
|---|---|---|---|---|---|
| Run Time (Min): | 9:30 | 10:00 | 14:00 | 15:00 | 9:00 |
| Schugi Rotor Speed | 3000 | 3000 | 3000 | 3000 | 3000 |
| (rpm) | | | | | |
| Schugi Motor Load | 1.1 | 1.1 | 1.1 | 1.1 | 1.1 |
| (amps) | | | | | |
| Knife Position | +5° | +5° | +5° | +5° | +5° |
| Nozzle Setup | N13 (3) | N13 (3) | N13 (3) | N13 (3) | N13 (1) |
| Nozzle Air Pressure (psi) | 65 | 65 | 65 | 65 | 65 |
| Feed Lot | SF 61043067 | SF 61043067 | SF 61043067 | SF 61043067 | SF 61043067 |
| | | | | | & 066 |
| Powder Feed Rate (lb/hr) | 200 | 200 | 200 | 200 | 100 |
| Liquid Binder | 6% | 6% | 4% | 4% | 4% |
| | Ethocel | Ethocel | Methocel | Methocel | Methocel |
| Liquid Feed Rate (lb/hr) | 31.5 | 31.5 | 49.5 | 49-32 | 25.7 |
| Coating Level (%) | 0.94 | 0.94 | 0.98 | 0.97-0.63% | 1.02 |

C. Fluid Bed Data; Model No. FBS-5.0

| | | | | | | |
|---|---|---|---|---|---|---|
| Inlet | Zone 1 | 130 | 133 | 150 | 150 | 150 |
| Air | Zone 2 | 136 | 136 | | 150 | 149 |
| Temp (° F.) | Zone 3 | 132 | 129 | | | 137 |
| Bed | Zone 1 | 55 | 56 | 90 | 73 | 74 |
| Temp | Zone 2 | 89 | 89 | | 73 | 76 |
| (° F.) | Zone 3 | 93 | 98 | | 73 | 74 |
| Air Out | Zone 1 | | | | | |
| Temp | Zone 2 | 69 | 66 | | | 72 |
| (° F.) | Zone 3 | | | | | |
| Air Flow | Zone 1 | | | | | 0.05 |
| Reading | Zone 2 | | | | | 0.05 |
| (in. H₂O) | Zone 3 | | | | | 0.01 |
| Total | Zone 1 | 4 | 5 | | | 2 |
| Pressure | Zone 2 | 3 | 3 | | | 2 |
| (in. H₂O) | Zone 3 | | | | | |
| Fluidizing | Zone 1 | 0 | 0 | 0 | 0 | |
| Velocity | Zone 2 | 0 | 0 | 0 | 0 | |
| (AFPM) | Zone 3 | 0 | 0 | 0 | 0 | |

D. Material Collected (lb)

| | | | | | | |
|---|---|---|---|---|---|---|
| Over Size | lb/hr | 159 | | 10.1 | | |
| (+20 M) | | | | | | |
| On Size | lb/hr | | | | | |
| (−20 M) | | | | | | |
| Baghouse | lb/hr | 41 | | | 50 | |
| Fines | | | | | | |
| FOA: | | | | | | |
| Vs(ft/min): | | 0.00 | 0.00 | 0.00 | 0.00 | 966.69 |
| | | 0.00 | 0.00 | 0.00 | 0.00 | 965.89 |
| | | 0.00 | 0.00 | 0.00 | 0.00 | 427.68 |
| gas | | 0.066474576 | 0.06613828 | 0.064295082 | 0.064295082 | 0.064295082 |
| density(lb/ft3): | | | | | | |
| | | 0.065805369 | 0.065805369 | 0.08526087 | 0.064295082 | 0.064400657 |
| | | 0.06625 | 0.066587436 | 0.08526087 | 0.08526087 | 0.065695142 |

TABLE 5
A. Schugi Agglomeration System

| | |
|---|---|
| Solid #1 Name | Succinate Fines |
| Solid #2 Name | |
| Liquid Name | |
| Screen Area (ft²) | 2.02 -Z1, Z2 1.03- Z3 |
| | Overflow Weir Z1/Z2/Z3: 8″/8″/8″ |
| | Underflow Weir Z1/Z2/Z3: All Closed |
| Screen Code Z1/Z2/Z3: | RAR Conidur |

| Run No.: | | | | |
|---|---|---|---|---|
| 7 | 7 | 7 | 7 | 7 |

B. Schugi Data; Model No. FX-100

| | | | | | |
|---|---|---|---|---|---|
| Run Time (Min): | 9:30 | 10:00 | 10:30 | 13:40 | 14:40 |
| Schugi Rotor Speed | 3000 | 3000 | 3000 | 3000 | 3000 |
| (rpm) | | | | | |
| Schugi Motor Load | 1.1 | 1.1 | 1.1 | 1.1 | 1.1 |
| (amps) | | | | | |
| Knife Position | +5° | +5° | +5° | +5° | +5° |
| Nozzle Setup | N13 (1) | N13 (1) | N13 (1) | N13 (1) | N13 (1) |
| Nozzle Air Pressure | 65 | 65 | 65 | 65 | 65 |
| (psi) | | | | | |
| Feed Lot | SF 61043067 | SF 61043067 | SF 61043067 | SF 61043067 | SF 61043067 |
| | & 066 | & 066 | & 066 | & 066 | & 066 |
| Powder Feed Rate | 100 | 100 | 100 | 100 | 100 |
| (lb/hr) | | | | | |
| Liquid Binder | 4% | 4% | 4% | 4% | 4% |
| | Methocel | Methocel | Methocel | Methocel | Methocel |
| Liquid Feed Rate (lb/hr) | 29.6 | 29.6 | 29.6 | 30 | 32 |
| Coating Level (%) | 1.17 | 1.17 | 1.17 | 1.19 | 1.26 |

C. Fluid Bed Data; Model No. FBS-5.0

| | | | | | | |
|---|---|---|---|---|---|---|
| Inlet | Zone 1 | 150 | 150 | 151 | 150 | 152 |
| Air | Zone 2 | 150 | 150 | 152 | 147 | 151 |
| Temp (° F.) | Zone 3 | 142 | 142 | 144 | 135 | 140 |
| Bed | Zone 1 | 74 | 74 | 74 | 75 | 83 |
| Temp | Zone 2 | 76 | 76 | 76 | 76 | 78 |
| (° F.) | Zone 3 | 74 | 74 | 74 | 74 | 76 |
| Air Out | Zone 1 | | | | | |
| Temp | Zone 2 | 72 | 72 | 73 | 73 | 76 |
| (° F.) | Zone 3 | | | | | |
| Air Flow | Zone 1 | 0.05 | 0.05 | 0.05 | 0.05 | 0.1 |
| Reading | Zone 2 | 0.05 | 0.05 | 0.05 | 0.05 | 0.05 |
| (in. H₂O) | Zone 3 | 0.01 | 0.01 | 0.01 | 0.01 | 0.01 |
| Total | Zone 1 | 2.4 | 2.4 | 2.5 | 2.8 | 3.8 |
| Pressure | Zone 2 | 2.4 | 2.4 | 2.5 | 2.8 | 3 |
| (in. H₂O) | Zone 3 | | | | | |
| Fluidizing | Zone 1 | 59 | 59 | 58 | 59 | 84 |
| Velocity | Zone 2 | 59 | 59 | 59 | 59 | 59 |
| (AFPM) | Zone 3 | 52 | 52 | 52 | 52 | 52 |

D. Material Collected (lb)

| | | | | | |
|---|---|---|---|---|---|
| Over Size | lb/hr | 159 | 10.1 | | 95.5 |
| (+20 M) | | | | | |
| On Size | lb/hr | | | | |
| (−20 M) | | | | | |
| Baghouse | lb/hr | 41 | | 50 | 4.5 |
| Fines | | | | | |

| | | | | | |
|---|---|---|---|---|---|
| FOA: | | | | | |
| Vs(ft/min): | 966.69 | 966.69 | 967.48 | 966.69 | 1369.34 |
| | 966.69 | 966.69 | 968.27 | 964.31 | 967.48 |
| | 429.47 | 429.47 | 430.18 | 426.97 | 428.76 |
| gas density | 0.064295082 | 0.064295082 | 0.064189853 | 0.064295082 | 0.064084967 |
| (lb/ft3): | | | | | |
| | 0.064295082 | 0.064295082 | 0.064084967 | 0.06461285 | 0.064189853 |
| | 0.065149502 | 0.065149502 | 0.064933775 | 0.065915966 | 0.065366667 |

### Example 2

### Characteristics of the METHOCEL® and ETHOCEL®-Coated Isoflavone

This example describes the characteristics of tocopheryl succinate prepared by methods described in Example 1. As shown in the Table 6, below, the product has slightly better bulk density using the two step process and shows better tablet formation characteristics compared to the product made using the one step fluidized bed process.

TABLE 6
A. Methocel-Coated Succinate

| | |
|---|---|
| Acid Value | 105.4 |
| Gardner Color | 4.8 |
| Tocopherol (mg/g) | 4.3 |
| Alpha Tocopheryl | 975.9 |
| Succinate (mg/g) | |
| Bulk Density (g/ml) | |
| *Tapped | 0.4420 |
| *Untapped | 0.3970 |

| Screens | % on | % thru |
|---|---|---|
| *20 | 0.04 | 99.96 |
| *80 | 77.20 | 22.76 |
| *100 | 7.58 | 15.18 |
| *120 | 4.70 | 10.48 |
| *140 | 1.82 | 8.66 |
| *200 | 4.79 | 3.87 |

B. Ethocel-Coated Succinate

| | |
|---|---|
| Acid Value | 105.3 |
| Gardner Color | 3.8 |
| Tocopherol (mg/g) | 5.1 |
| Alpha Tocopheryl | 964.2 |
| Succinate (mg/g) | |
| Bulk Density (g/ml) | |
| *Tapped | 0.4104 |
| *Untapped | 0.3754 |

| Screens | % on | % thru |
|---|---|---|
| *20 | 0.07 | 99.93 |
| *80 | 85.58 | 14.35 |
| *100 | 7.98 | 6.37 |
| *120 | 3.93 | 2.44 |
| *140 | 2.31 | 0.13 |
| *200 | 0.12 | 0.01 |
| EtOH (ppm) | 1 | |

Having now fully described the present invention in some detail by way of illustration and example for purposes of clarity of understanding, it will be obvious to one of ordinary skill in the art that the same can be practiced by modifying or changing the invention with a wide and equivalent range of conditions, formulations and other parameters thereof, and that such modifications are intended to be encompassed within the scope of the appended claims.

All publications, patents and patent applications mentioned in this specification are indicative of the level of skill of those skilled in the art to which this invention pertains, and are herein incorporated by reference to the same extent as if each individual publication, patent or patent application was specifically and individually indicated to be incorporated herein by reference.

1. A process for producing a coated tocopherol succinate, comprising:
(a) dispersing a binder composition in a solvent to form a binder solution;
(b) passing tocopherol succinate powder through the binder solution, where the binder solution is in an atomized state, to produce wetted tocopherol succinate;
(c) passing the wetted tocopherol succinate through a region of turbulent gas to form agglomerated tocopherol succinate; and
(d) evaporating the solvent from the agglomerated tocopherol succinate;
thereby forming dried coated tocopherol succinate.

2. The process of claim 1, further comprising screening the dried coated tocopherol succinate.

3. The process of claim 2, wherein particles over 20 mesh in size are removed.

4. The process of claim 1, wherein the binder composition is at least one material selected from the group consisting of cellulose, cellulose derivatives, maltodextrin, alginic acid derivatives, calcium lactate, gum arabic, gelatin, sugar, sugar alcohols, glycerol, starch, modified starch, pregelatinized starch, polyvinylpyrrolidones, stearic acid, gum acacia, and hydrogenated vegetable oil.

5. The process of claim 4, wherein the cellulose derivative is at least one material selected from the group consisting of ethylcellulose, methylcellulose and hydroxypropylmethylcellulose.

6. The process of claim 1, wherein the solvent is selected from the group consisting of water, C1-C3 alcohol, and a mixture of water and C1-C3 alcohol.

7. The process of claim 6, wherein at least one alcohol is selected from the group consisting of ethanol, isopropanol, methanol, or a mixture thereof.

8. The process of claim 1, wherein the solvent is evaporated by a fluid bed dryer.

9. The process of claim 1, wherein the coated tocopherol succinate is a free-flowing powder.

10. The process of claim 1, wherein the binder solution is about 0.5% to about 10.0% hydroxypropylmethylcellulose in water.

11. The process of claim 1, wherein the binder solution is about 3.0% to about 4.0% hydroxypropylmethylcellulose in water.

12. A coated tocopherol succinate, made by the process of:
(a) dispersing a binder composition in a solvent to form a binder solution;
(b) passing tocopherol succinate in powder form through the binder solution, where the binder solution is in an atomized state, to produce wetted tocopherol succinate;
(c) passing the wetted tocopherol succinate through a region of turbulent gas to form agglomerated tocopherol succinate; and
(d) evaporating the solvent from the agglomerated tocopherol succinate.

13. The coated tocopherol succinate of claim 12, where the process further comprises screening the dried coated tocopherol succinate.

14. The coated tocopherol succinate of claim 13, wherein particles over 20 mesh in size are removed.

15. The coated tocopherol succinate of claim 12, wherein the binder composition is at least one material selected from the group consisting of cellulose, cellulose derivatives, maltodextrin, alginic acid derivatives, calcium lactate, gum arabic, gelatin, sugar, sugar alcohols, glycerol, starch, modified starch, pregelatinized starch, polyvinylpyrrolidones, stearic acid, gum acacia, and hydrogenated vegetable oil.

16. The coated tocopherol succinate of claim 15, wherein the cellulose derivative is at least one material selected from the group consisting of ethylcellulose, methylcellulose and hydroxypropylmethylcellulose.

17. The coated tocopherol succinate of claim 12, wherein the solvent is selected from the group consisting of water, C1-C3 alcohol, and a mixture of water and C1-C3 alcohol.

18. The coated tocopherol succinate of claim 17, wherein at least one alcohol is selected from the group consisting of ethanol, isopropanol, methanol, or a mixture thereof.

19. The coated tocopherol succinate of claim 12, wherein the solvent is evaporated by a fluid bed dryer.

20. The coated tocopherol succinate of claim 12, wherein the coated tocopherol succinate is a free-flowing powder.

21. The coated tocopherol succinate of claim 12, wherein the binder solution is about 0.5% to about 10.0% hydroxypropylmethylcellulose in water.

22. The coated tocopherol succinate of claim 12, wherein the binder solution is about 3.0% to about 4.0% hydroxypropylmethylcellulose in water.

23. A process for producing a coated phytoestrogen composition, comprising:
(a) dispersing a binder composition in a solvent to form a binder solution;
(b) passing phytoestrogen composition in powder form through the binder solution, where the binder solution is in an atomized state, to produce wetted phytoestrogen composition;
(c) passing the wetted phytoestrogen composition through a region of turbulent gas to form agglomerated phytoestrogen composition; and
(d) evaporating the solvent from the agglomerated phytoestrogen composition;
thereby forming dried coated phytoestrogen composition.

24. The process of claim 23, further comprising screening the dried coated phytoestrogen composition.

25. The process of claim 24, wherein particles over 20 mesh in size are removed.

26. The process of claim 23, wherein the binder composition is at least one material selected from the group consisting of cellulose, cellulose derivatives, maltodextrin, alginic acid derivatives, calcium lactate, gum arabic, gelatin, sugar, sugar alcohols, glycerol, starch, modified starch, pregelatinized starch, polyvinylpyrrolidones, stearic acid, gum acacia, and hydrogenated vegetable oil.

27. The process of claim 26, wherein the cellulose derivative is at least one material selected from the group consisting of ethylcellulose, methylcellulose and hydroxypropylmethylcellulose.

28. The process of claim 23, wherein the solvent is selected from the group consisting of water, C1-C3 alcohol, and a mixture of water and C1-C3 alcohol.

29. The process of claim 28, wherein at least one alcohol is selected from the group consisting of ethanol, isopropanol, methanol, or a mixture thereof.

30. The process of claim 23, wherein the solvent is evaporated by a fluid bed dryer.

31. The process of claim 23, wherein the phytoestrogen is an isoflavone.

32. The process of claim 31, wherein the isoflavone is selected from the group consisting of genistein, daidzein, glycitein, biochanin A, equol, formononetin, and o-desmethylangolensin.

33. The process of claim 31, wherein the isoflavone is selected from the group consisting of natural derivatives of genistein, daidzein, glycitein, biochanin A, equol, formononetin, and o-desmethylangolensin.

34. The process of claim 31, wherein the isoflavone is selected from the group consisting of chemical derivatives of genistein, daidzein, glycitein, biochanin A, equol, formononetin, and o-desmethylangolensin.

35. The process of claim 34, wherein the coated phytoestrogen composition is a free-flowing powder.

36. The process of claim 34, wherein the binder solution is about 0.5% to about 10.0% hydroxypropylmethylcellulose in water.

37. The process of claim 34, wherein the binder solution is about 3.0% to about 4.0% hydroxypropylmethylcellulose in water.

38. A coated phytoestrogen composition, made by the process of:
(a) dispersing a binder composition in a solvent to form a binder solution;
(b) passing phytoestrogen composition in powder form through the binder solution, where the binder solution is in an atomized state, to produce wetted phytoestrogen composition;
(c) passing the wetted phytoestrogen composition through a region of turbulent gas to form agglomerated phytoestrogen composition; and
(d) evaporating the solvent from the agglomerated phytoestrogen composition.

39. The coated phytoestrogen composition of claim 38, where the process further comprises screening the dried coated phytoestrogen composition.

40. The coated phytoestrogen composition of claim 39, wherein particles over 20 mesh in size are removed.

41. The coated phytoestrogen composition of claim 38, wherein the binder composition is at least one material selected from the group consisting of cellulose, cellulose derivatives, maltodextrin, alginic acid derivatives, calcium lactate, gum arabic, gelatin, sugar, sugar alcohols, glycerol, starch, modified starch, pregelatinized starch, polyvinylpyrrolidones, stearic acid, gum acacia, and hydrogenated vegetable oil.

42. The coated phytoestrogen composition of claim 41, wherein the cellulose derivative is at least one material selected from the group consisting of ethylcellulose, methylcellulose and hydroxypropylmethylcellulose.

43. The coated phytoestrogen composition of claim 38, wherein the solvent is selected from the group consisting of water, C1-C3 alcohol, and a mixture of water and C1-C3 alcohol.

44. The coated phytoestrogen composition of claim 43, wherein at least one alcohol is selected from the group consisting of ethanol, isopropanol, methanol, or a mixture thereof.

45. The coated phytoestrogen composition of claim 38, wherein the solvent is evaporated by a fluid bed dryer.

46. The coated phytoestrogen composition of claim 38, wherein the phytoestrogen is an isoflavone.

47. The coated phytoestrogen composition of claim 46, wherein the isoflavone is selected from the group consisting of genistein, daidzein, glycitein, biochanin A, equol, formononetin, and o-desmethylangolensin.

48. The coated phytoestrogen composition of claim 46, wherein the isoflavone is selected from the group consisting of natural derivatives of genistein, daidzein, glycitein, biochanin A, equol, formononetin, and o-desmethylangolensin.

49. The coated phytoestrogen composition of claim 46, wherein the isoflavone is selected from the group consisting of chemical derivatives of genistein, daidzein, glycitein, biochanin A, equol, formononetin, and o-desmethylangolensin.

50. The coated phytoestrogen composition of claim 38, wherein the coated phytoestrogen composition is a free-flowing powder.

51. The coated phytoestrogen composition of claim 38, wherein the binder solution is about 0.5% to about 10.0% hydroxypropylmethylcellulose in water.

52. The coated phytoestrogen composition of claim 38, wherein the binder solution is about 3.0% to about 4.0% hydroxypropylmethylcellulose in water.
